(19) United States
(12) Patent Application Publication
(10) Pub. No.: US 2009/0023671 A1
(43) Pub. Date: Jan. 22, 2009

(54) Rnai Agents for Maintenance of Stem Cells

(76) Inventors: Sarah J. Brashears, McLean, VA (US); Elisabeth Evertsz, San Jose, CA (US)

Correspondence Address: PATTON BOGGS LLP, 8484 WESTPARK DRIVE, SUITE 900, MCLEAN, VA, 22102, US

(21) Appl. No.: 11/794,726

(22) PCT Filed: Jan. 4, 2006
(86) PCT No.: PCT/US06/00091

§ 371 Date: Sep. 16, 2008

Related U.S. Application Data
(60) Provisional application No. 60/641,929, filed on Jan. 6, 2005.

(51) Int. Cl. A61K 31/7105; C12N 5/10; A61P 43/00

(52) U.S. Cl. 514/44; 435/377; 435/366

(57) ABSTRACT

The present invention provides compositions and methods suitable for delivering RNAi agents against genetic targets in stem cells so as to direct cell growth and differentiation.

### BACKGROUND OF THE INVENTION

Utilization of double-stranded RNA to inhibit gene expression in a sequence-specific manner has revolutionized the drug discovery industry. In mammals, RNA interference, or RNAi, is mediated by 15- to 49-nucleotide long, double-stranded RNA molecules referred to as small interfering RNAs (RNAi agents). RNAi agents can be synthesized chemically or enzymatically outside of cells and subsequently delivered to cells (see, e.g., Fire, et al., Nature, 391:806-11 (1998); Tuschl, et al., Genes and Dev., 13:3191-97 (1999); and Elbashir, et al, Nature, 411:494-498 (2001)); or can be expressed in vivo by an appropriate vector in cells (see, e.g., U.S. Pat. No. 6,573,099).

In vivo delivery of unmodified RNAi agents as an effective therapeutic for use in humans faces a number of technical hurdles. First, due to cellular and serum nucleases, the half life of RNA injected in vivo is only about 70 seconds (see, e.g., Kurreck, Eur. J. Bioch. 270:1628-44 (2003)). Efforts have been made to increase stability of injected RNA by the use of chemical modifications; however, there are several instances where chemical alterations led to increased cytotoxic effects. In one specific example, cells were intolerant to doses of an RNAi duplex in which every second phosphate was replaced by phosphorothioate (Harborth, et al., Antisense Nucleic Acid Drug Rev. 13(2): 83-105 (2003)). Still on going efforts are directed to find ways to delivery unmodified or modified RNAi agents so as to provide tissue-specific delivery, as well as deliver the RNAi agents in amounts sufficient to elicit a therapeutic response but that are not toxic.

Other options being explored for RNAi delivery include the use of viral-based and non-viral based vector systems that can infect or otherwise transfect target cells, and deliver and express RNAi molecules in situ. Often, small RNAs are transcribed as short hairpin RNA (shRNA) precursors from a viral or non-viral vector backbone. Once transcribed, the shRNA are processed by the enzyme Dicer into the appropriate active RNAi agents. Viral-based delivery approaches attempt to exploit the targeting properties of viruses to generate tissue specificity and once appropriately targeted, rely upon the endogenous cellular machinery to generate sufficient levels of the RNAi agents to achieve a therapeutically effective dose.

One useful application of RNAi therapeutics is in the maintainence and proliferation of hematopoietic stem cells. Mammalian blood cells provide for an extraordinarily diverse range of activities. The blood cells are divided into several lineages, including lymphoid, myeloid and erythroid. The lymphoid lineage, comprising B-cells and T-cells, provides for the production of antibodies, regulation of the cellular immune system, detection of foreign agents in the blood, detection of cells foreign to the host, and the like. The myeloid lineage, which includes monocytes, granulocytes, megakaryocytes as well as other cells, monitors for the presence of foreign bodies in the blood stream, provides protection against neoplastic cells, scavenges foreign materials in the blood stream, produces platelets, and the like. The erythroid lineage provides the red blood cells, which act as oxygen carriers.

Despite the diversity of the nature, morphology, characteristics and function of the blood cells, it is presently believed that there is a single progenitor hematopoietic stem cell, which is capable of self regeneration and by exposure to growth factors becomes dedicated to a specific lineage. The hematopoietic stem cell (HSC) population constitutes only a small percentage of the total number of white blood cells in bone marrow

There is a strong interest in preventing the differentiation of stem cells and/or dedication of stem cells to particular lineages and controlling of stem cell proliferation. The availability of greater amounts of stem cells would be extremely useful in bone marrow transplantation, as well as transplantation of other organs in association with the transplantation of bone marrow. Stem cells are important targets for gene therapy, where the inserted genes promote the health of the individual into whom the stem cells are transplanted. In addition, the ability to isolate the stem cell may serve in the treatment of lymphomas and leukemias, as well as other neoplastic conditions, e.g., breast cancer.

Clinical and basic investigators share the same fundamental problem—limited ability to grow and expand the numbers of human HSCs. Clinicians repeatedly see that larger numbers of cells in stem cell grafts have a better chance of survival in a patient than do smaller numbers of cells. The limited number of cells available from a placenta and umbilical cord blood transplant currently means that cord blood banks are useful to pediatric but not adult patients. Ability to expand numbers of human HSCs in vivo or in vitro would clearly be an enormous boost to all current and future medical uses of HSC transplantation.

Thus, there is a need in the art to develop stable, effective RNAi methods promote stem cell proliferation and to inhibit apoptosis and differentiation in order to provide and expanded number of stem cells for therapy and research. The present invention satisfies this need in the art.

### SUMMARY OF THE INVENTION

The present invention provides stable, effective siRNA and ddRNAi reagents and methods for use thereof to control the differentiation and proliferation of stem cells by altering the level of expression of one or more transcriptionally active genetic regions that are directly or indirectly associated with the differentiation and proliferation of stem cells.

The present invention provides a method for controlling differentiation and proliferation of stem cells together with genetic agents for use therewith, as well as genetically modified cells comprising the genetic agents. The present invention would allow for the in vitro proliferation of stem cells for research purposes. Another aspect of this invention would allow for the ex vivo stimulation of stem cells to differentiate to a particular path of progenitor cells. The present invention is predicated in part on the use of genetic agents that facilitate gene silencing via RNAi to downregulate or silence one or more transcriptionally active genetic regions directly or indirectly associated with the differentiation and proliferation of stem cells. Such transcriptionally active regions are also referred to herein as “stem cell associated genetic targets” or “SCATs”. siRNA and ddRNAi-mediated silencing of one or more SCATs effects control of the proliferation of stem cells in a subject or cell culture.

Accordingly, one aspect of the present invention contemplates a method for promoting cell growth and inhibiting differentiation in a subject or cell culture, said method comprising administering to said subject or cell culture a genetic construct comprising at least one ddRNAi expression cassette which encodes an RNA molecule comprising a nucleotide sequence which is at least 70% identical to at least part of a nucleotide sequence comprising a SCAT or a derivative, ortholog or homolog thereof and which delays, represses or otherwise reduces the expression of the SCAT in said subject. In one aspect of the present invention the stem cells to be affected can be comprised of stem cell lines known in the art such as hematopoietic stem cells acquired from bone marrow transplants, apheresis procedures, umbilical cord blood, or any other source known to one of skill in the art.

In another aspect, the present invention provides genetically modified cells comprising a ddRNAi expression cassette that expresses a ddRNAi agent that delays, represses, or otherwise reduces the expression of one or more transcriptionally active genetic regions that are directly or indirectly associated with the differentiation and proliferation of stem cells. Preferably the cell is a mammalian cell, even more preferably the cell is a primate or rodent cell and most preferably the cell is a human or mouse cell. Furthermore, in yet another aspect, the present invention provides a multicellular structure comprising one or more genetically modified cells of the present invention. Multi-cellular structures include, inter alia, include a tissue, organ or complete organism.

Yet another aspect of the present invention contemplates a method for promoting cell growth and inhibiting differentiation in a subject or cell culture, said method comprising administering to said subject or cell culture an siRNA which encodes an RNA molecule comprising a nucleotide sequence which is at least 70% identical to at least part of a nucleotide sequence comprising a SCAT or a derivative, ortholog or homolog thereof and which delays, represses or otherwise reduces the expression of the SCAT in said subject.

Other objects and advantages of the present invention will be apparent from the detailed description that follows.

### BRIEF DESCRIPTION OF THE DRAWINGS

So that the manner in which the above recited features, advantages and objects of the present invention are attained and can be understood in detail, a more particular description of the invention, briefly summarized above, may be had by reference to the embodiments that are illustrated in the appended drawings. It is to be noted, however, that the appended drawings illustrate only certain embodiments of this invention and are therefore not to be considered limiting of its scope, for the present invention may admit to other equally effective embodiments.

FIGS. 1A, 1B and 1C are simplified block diagrams of three embodiments of methods for delivering RNAi agents to modulating stem cell growth and/or differentiation according to the present invention.

FIGS. 2A and 2B show two embodiments of single-expression RNAi cassettes, and FIGS. 2C and 2D show two embodiments of multiple-expression RNAi cassettes.

FIGS. 3A and 3B show two embodiments of multiple expression cassettes that code for RNAi agents initially expressed as shRNA precursors, and FIGS. 3C and 3D show two embodiments of multiple expression cassettes that code for RNAi agents that are not expressed as shRNA precursors.

FIGS. 4A and 4B show alternative methods for producing viral particles for delivery of ddRNAi agents to cells.

### DETAILED DESCRIPTION

Before the present compositions and methods are described, it is to be understood that this invention is not limited to the particular methodology, products, apparatus and factors described, as such methods, apparatus and formulations may, of course, vary. It is also to be understood that the terminology used herein is for the purpose of describing particular embodiments only, and is not intended to limit the scope of the present invention, which will be limited only by appended claims.

As used herein, the singular forms “a,” “an,” and “the” include plural referents unless the context clearly dictates otherwise. Thus, for example, reference to “a factor” refers to one or mixtures of factors, and reference to “the method of production” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference, without limitation, for the purpose of describing and disclosing devices, formulations and methodologies which are described in the publication and which might be used in connection with the presently described invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

The present invention is directed to innovative, robust genetic compositions and methods to promote stem cell growth and/or inhibit cell differentiation. The compositions and methods provide stable, lasting and regulatable inhibition of a target gene or gene family.

Generally, conventional methods of molecular biology, microbiology, recombinant DNA techniques, cell biology, and virology within the skill of the art are employed in the present invention. Such techniques are explained fully in the literature, see, e.g., Maniatis, Fritsch & Sambrook, Molecular Cloning: A Laboratory Manual (1982); DNA Cloning: A Practical Approach, Volumes I and II (D. N. Glover, ed. 1985); Oligonucleotide Synthesis (M. J. Gait, ed. 1984); Nucleic Acid Hybridization (B. D. Hames & S. J. Higgins, eds. (1984)); Animal Cell Culture (R. I. Freshney, ed. 1986); and RNA Viruses: A practical Approach, (Alan, J. Cann, Ed., Oxford University Press, 2000).

A “vector” is a replicon, such as plasmid, phage, viral construct or cosmid, to which another DNA segment may be attached. Vectors are used to transduce and express the DNA segment in cells. As used herein, the terms “vector”, “construct”, “ddRNAi expression vector” or “ddRNAi expression construct” may include replicons such as plasmids, phage, viral constructs, cosmids, Bacterial Artificial Chromosomes (BACs), Yeast Artificial Chromosomes (YACs) Human Artificial Chromosomes (HACs) and the like into which one or more ddRNAi expression cassettes may be or are ligated.

A “promoter” or “promoter sequence” is a DNA regulatory region capable of binding RNA polymerase in a cell and initiating transcription of a polynucleotide or polypeptide coding sequence such as messenger RNA, ribosomal RNAs, small nuclear of nucleolar RNAs or any kind of RNA transcribed by any class of any RNA polymerase.

A cell has been “transformed”, “transduced” or “transfected” by an exogenous or heterologous nucleic acid or vector when such nucleic acid has been introduced inside the cell, for example, as a complex with transfection reagents or packaged in viral particles. The transforming DNA may or may not be integrated (covalently linked) into the genome of the cell. With respect to eukaryotic cells, a stably transformed cell is one in which the transforming DNA has become integrated into a host cell chromosome or is maintained extra-chromosomally so that the transforming DNA is inherited by daughter cells during cell replication or is a non-replicating, differentiated cell in which a persistent episome is present.

The term “RNA interference” or “RNAi” refers generally to a process in which a double-stranded RNA molecule changes the expression of a nucleic acid sequence with which the double-stranded RNA molecule shares substantial or total homology. The term “RNAi agent” refers to an RNA sequence—either a modified or unmodified synthetic oligoribonucleontide (“siRNA”) or a DNA-delivered RNAi (i.e., an RNAi agent that is transcribed from a vector, also referred to as “ddRNAi”)—that elicits RNAi. The terms “short hairpin RNA” or “shRNA” refer to an RNA structure having a duplex region and a loop region. In some embodiments of the present invention, ddRNAi agents are expressed initially as shRNAs. The term “RNAi expression cassette” refers to a cassette according to embodiments of the present invention having at least one [promoter-RNAi agent-terminator] unit. The term “multiple promoter RNAi expression cassette” refers to an RNAi expression cassette comprising two or more [promoter-RNAi agent-terminator] units. The terms “RNAi expression construct” or “RNAi expression vector” refer to vectors containing an RNAi expression cassette.

“Derivatives” of a gene or nucleotide sequence refers to any isolated nucleic acid molecule that contains significant sequence similarity to the gene or nucleotide sequence or a part thereof. In addition, “derivatives” include such isolated nucleic acids containing modified nucleotides or mimetics of naturally-occurring nucleotides.

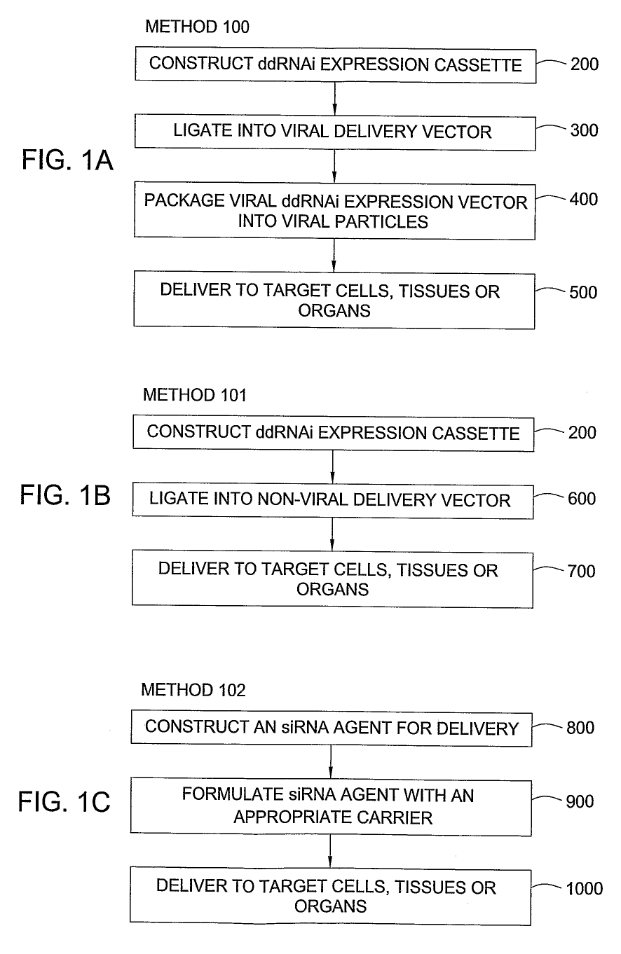

FIGS. 1A, 1B and 1C are simplified flow charts showing the steps of methods according to three embodiments of the present invention in which an RNAi agent according to the present invention may be used. Method 100 of FIG. 1A includes a step 200 in which a ddRNAi expression cassette is constructed. Such a ddRNAi expression cassette most often will include at least one promoter, a ddRNAi sequence to be expressed, and at least one terminator. Various configurations of such ddRNAi expression cassettes are described in detail infra. In step 300, the ddRNAi expression cassette is ligated into viral delivery vector, and at step 400, the ddRNAi viral delivery vector is packaged into viral particles. Finally, at step 500, the viral particles are delivered to target cells, tissues or organs. FIG. 1B shows a method 101 where again, at step 200, a ddRNAi expression cassette is constructed. In Figure B, however, the ddRNAi expression cassette is ligated into a non-viral delivery vector at step 600. Then, at step 700, the non-viral ddRNAi delivery vector is delivered to target cells, tissues or organs. FIG. 1C shows a method 102 where at step 800, an siRNA agent is constructed for delivery. At step 900, the siRNA is formulated with an appropriate carrier for delivery. Finally, at step 1000, the siRNA agent/carrier is delivered to target cells, tissues, or organs.

RNAi agents according to the present invention can be generated synthetically or enzymatically by a number of different protocols known to those skilled in the art and purified using standard recombinant DNA techniques as described in, for example, Sambrook et al., Molecular Cloning: A Laboratory Manual, 2nd Ed., Cold Spring Harbor Press, Cold Spring Harbor, N.Y. (1989), and under regulations described in, e.g., United States Dept. of HHS, National Institute of Health (NIH) Guidelines for Recombinant DNA Research.

RNAi agents may comprise either siRNAs (synthetic RNAs) or DNA-directed RNAs (ddRNAs). siRNAs may be manufactured by methods known in the art such as by typical oligonucleotide synthesis, and often will incorporate chemical modifications to increase half life and/or efficacy of the siRNA agent, and/or to allow for a more robust delivery formulation. Many modifications of oligonucleotides are known in the art. For example, U.S. Pat. No. 6,620,805 discloses an oligonucleotide that is combined with a macrocycle having a net positive charge such as a porphyrin; U.S. Pat. No. 6,673,611 discloses various formulas; U.S. Publ. Nos. 2004/0171570, 2004/0171032, and 2004/0171031 disclose oligomers that include a modification comprising a polycyclic sugar surrogate; such as a cyclobutyl nucleoside, cyclopentyl nucleoside, proline nucleoside, cyclohexene nucleoside, hexose nucleoside or a cyclohexane nucleoside; and oligomers that include a non-phosphorous-containing internucleoside linkage; U.S. Publ. No 2004/0171579 discloses a modified oligonucleotide where the modification is a 2′ substituent group on a sugar moiety that is not H or OH; U.S. Publ. No. 2004/0171030 discloses a modified base for binding to a cytosine, uracil, or thymine base in the opposite strand comprising a boronated C and U or T modified binding base having a boron-containing substituent selected from the group consisting of —BH₂CN, —BH₃, and —BH₂COOR, wherein R is C1 to C18 alkyl; U.S. Publ. No. 2004/0161844 discloses oligonucleotides having phosphoramidate internucleoside linkages such as a 3′aminophosphoramidate, aminoalkylphosphoramidate, or aminoalkylphosphorthioamidate internucleoside linkage; U.S. Publ. No. 2004/0161844 discloses yet other modified sugar and/or backbone modifications, where in some embodiments, the modification is a peptide nucleic acid, a peptide nucleic acid mimic, a morpholino nucleic acid, hexose sugar with an amide linkage, cyclohexenyl nucleic acid (CeNA), or an acyclic backbone moiety; U.S. Publ. No. 2004/0161777 discloses oligonucleotides with a 3′ terminal cap group; U.S. Publ. No. 2004/0147470 discloses oligomeric compounds that include one or more cross-linkages that improve nuclease resistance or modify or enhance the pharmacokinetic and phamacodynamic properties of the oligomeric compound where such cross-linkages comprise a disulfide, amide, amine, oxime, oxyamine, oxyimine, morpholino, thioether, urea, thiourea, or sulfonamide moiety; U.S. Publ. No. 2004147023 discloses a gapmer comprising two terminal RNA segments having nucleotides of a first type and an internal RNA segment having nucleotides of a second type where nucleotides of said first type independently include at least one sugar substituent where the sugar substituent comprises a halogen, amino, trifluoroalkyl, trifluoroalkoxy, azido, aminooxy, alkyl, alkenyl, alkynyl, O—, S—, or N(R*)-alkyl; O—, S—, or N(R*)-alkenyl; O—, S— or N(R*)-alkynyl; O—, S- or N-aryl, O—, S—, or N(R*)-aralkyl group; where the alkyl, alkenyl, alkynyl, aryl or aralkyl may be a substituted or unsubstituted alkyl, alkenyl, alkynyl, aralkyl; and where, if substituted, the substitution is an alkoxy, thioalkoxy, phthalimido, halogen, amino, keto, carboxyl, nitro, nitroso, cyano, trifluoromethyl, trifluoromethoxy, imidazole, azido, hydrazino, aminooxy, isocyanato, sulfoxide, sulfone, disulfide, silyl, heterocycle, or carbocycle group, or an intercalator, reporter group, conjugate, polyamine, polyamide, polyalkylene glycol, or a polyether of the formula (—O-alkyl)ₘ, where m is 1 to about 10; and R* is hydrogen, or a protecting group; or U.S. Publ. No. 2004/0147022 disclosing an oligonucleotide with a modified sugar and/or backbone modification, such as a 2′-OCH₃ substituent group on a sugar moiety.

Alternatively, DNA-directed RNAi (ddRNAi) agents may be employed. ddRNAi agents comprise an expression cassette, most often containing at least one promoter, at least one ddRNAi sequence and at least one terminator in a viral or non-viral vector backbone. For example, in one preferred embodiment, the ddRNAi expression cassette comprises a nucleic acid molecule comprising the general structure (I):

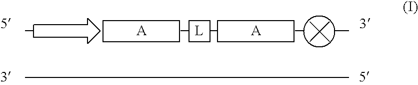

wherein:
[symbol] represents a promoter sequence;
[symbol] represents a ddRNAi targeting sequence comprising at least 10 nucleotides, wherein said sequence is at least 70% identical to a SCAT sequence or part thereof;
[symbol] represents a sequence of 10 to 30 nucleotides wherein at least 10 contiguous nucleotides of A′ comprise a reverse complement of the nucleotide sequence represented by A;
[symbol] represents a “loop” encoding structure comprising a sequence of 5 to 20 non-self-complementary nucleotides that serve to separate A and A′; and
[symbol] represents a terminator sequence.
   The ddRNAi agent generated by the expression of the ddRNAi expression cassette represented by general structure (I) comprises a stem-loop structured precursor (shRNA) in which the ends of the double-stranded RNA are connected by a single-stranded, linker RNA. The length of the single-stranded loop portion of the shRNA may be 5 to 20 bp in length, and is preferably 5 to 9 bp in length. Accordingly, in a preferred embodiment, L in general structure (I) comprises 5, 6, 7, 8 or 9 non-self-complementary nucleotides.

In another embodiment, the ddRNAi expression cassette comprises a nucleic acid molecule of the general structure (II):

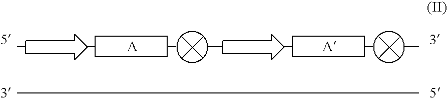

wherein:
[symbol] represents promoter sequence;
[symbol] represents a ddRNAi targeting sequence comprising at least 10 nucleotides, wherein said sequence is at least 70% identical to a SCAT sequence or part thereof;
[symbol] represents a sequence of 10 to 30 nucleotides wherein at least 10 contiguous nucleotides of A′ comprise a reverse complement of the nucleotide sequence represented by A; and
[symbol] represents a terminator sequence.

In yet another embodiment, the ddRNAi expression cassette comprises a nucleic acid molecule of the general structure (III):

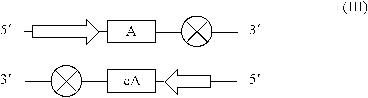

wherein:
[symbol] represents a promoter sequence;
[symbol] represents a ddRNAi targeting sequence comprising at least 10 nucleotides, wherein said sequence is at least 70% identical to a SCAT sequence or part thereof;
[symbol] represents a nucleic acid sequence complementary to A; and
[symbol] represents a terminator sequence.

In yet another preferred embodiment, the ddRNAi expression cassette comprises a nucleic acid molecule of the general structure (IV):

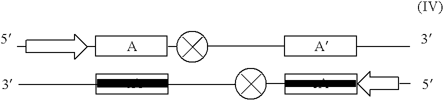

wherein:
[symbol] represents a promoter sequence;
[symbol] represents a ddRNAi targeting sequence comprising at least 10 nucleotides, wherein said sequence is at least 70% identical to a SCAT sequence or part thereof;
[symbol] represents a nucleic acid sequence complementary to A; and
[symbol] represents a terminator sequence.

Although the ddRNAi expression cassettes represented by general structures (I), (II), (III) and (IV) represent preferred embodiments of the invention, the present invention is in no way limited to these particular general structures. As would be evident to one of skill in the art, the above structures may be modified while retaining functionality. For example, the elements of the cassettes may be separated by one or more nucleotide residues. Furthermore, elements which are present on complementary strands, such as the terminator and promoter elements shown in structures (III) and (IV) may overlap or may be discreet. For example, the terminator elements shown in structure (III) may occur within the complementary strand of the promoter element or may be upstream or downstream of this region. Other modifications which would be evident to one of skill in the art and which do not materially effect the functioning of the cassette in encoding a dsRNA structure may also be made and such modified cassettes are within the scope of the present invention.

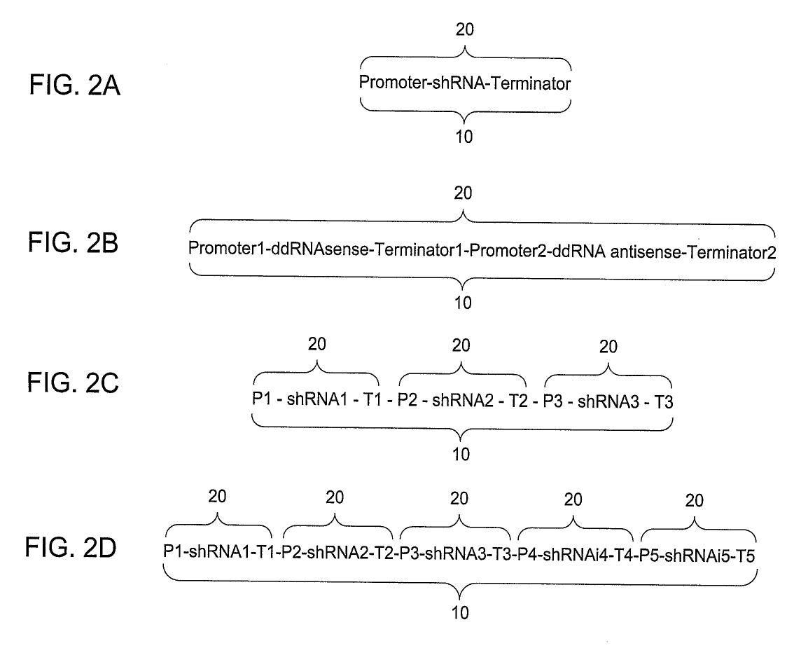

FIGS. 2A through 2D show additional examples of ddRNAi expression cassettes. FIGS. 2A and 2B are simplified schematics of single-promoter RNAi expression cassettes according to embodiments of the present invention. FIG. 2A shows an embodiment of a single RNAi expression cassette (10) comprising one promoter/RNAi/terminator component (shown at 20), where the ddRNAi agent is expressed initially as a short hairpin (shRNA). FIG. 2B shows an embodiment of a single RNAi expression cassette (10) with one promoter/RNAi/terminator component (shown at 20), where the sense and antisense components of the ddRNAi agent are expressed separately from different promoters.

FIGS. 2C and 2D are simplified schematics of multiple-promoter RNAi expression cassettes according to embodiments of the present invention. FIG. 2C shows an embodiment of a multiple-promoter RNAi expression cassette (10) comprising three promoter/RNAi/terminator components (shown at 20), and FIG. 2D shows an embodiment of a multiple-promoter expression cassette (10) with five promoter/RNAi/terminator components (shown at 20). P1, P2, P3, P4 and P5 represent promoter elements. RNAi1, RNAi2, RNAi3, RNAi4 and RNAi5 represent sequences for five different ddRNAi agents. T1, T2, T3, T4, and T5 represent termination elements. The multiple-promoter RNAi expression cassettes according to the present invention may contain two or more promoter/RNAi/terminator components where the number of promoter/RNAi/terminator components included in any multiple-promoter RNAi expression cassette is limited by, e.g., packaging size of the delivery system chosen (for example, some viruses, such as AAV, have relatively strict size limitations); cell toxicity, and maximum effectiveness (i.e. when, for example, expression of four ddRNAi agents is as effective therapeutically as the expression of ten ddRNAi agents).

When employing a multiple promoter RNAi expression cassette, the two or more ddRNAi agents in the promoter/RNAi/terminator components comprising a cassette all have different sequences; that is RNAi1, RNAi2, RNAi3, RNAi4 and RNAi5 are all different from one another. However, the promoter elements in any cassette may be the same (that is, e.g., the sequence of two or more of P1, P2, P3, P4 and P5 may be the same); all the promoters within any cassette may be different from one another; or there may be a combination of promoter elements represented only once and promoter elements represented two times or more within any cassette. Similarly, the termination elements in any cassette may be the same (that is, e.g., the sequence of two or more of T1, T2, T3, T4 and T5 may be the same, such as contiguous stretches of 4 or more T residues); all the termination elements within any cassette may be different from one another; or there may be a combination of termination elements represented only once and termination elements represented two times or more within any cassette. Preferably, the promoter elements and termination elements in each promoter/RNAi/terminator component comprising any cassette are all different to decrease the likelihood of DNA recombination events between components and/or cassettes. Further, in a preferred embodiment, the promoter element and termination element used in each promoter/RNAi/terminator component are matched to each other; that is, the promoter and terminator elements are taken from the same gene in which they occur naturally.

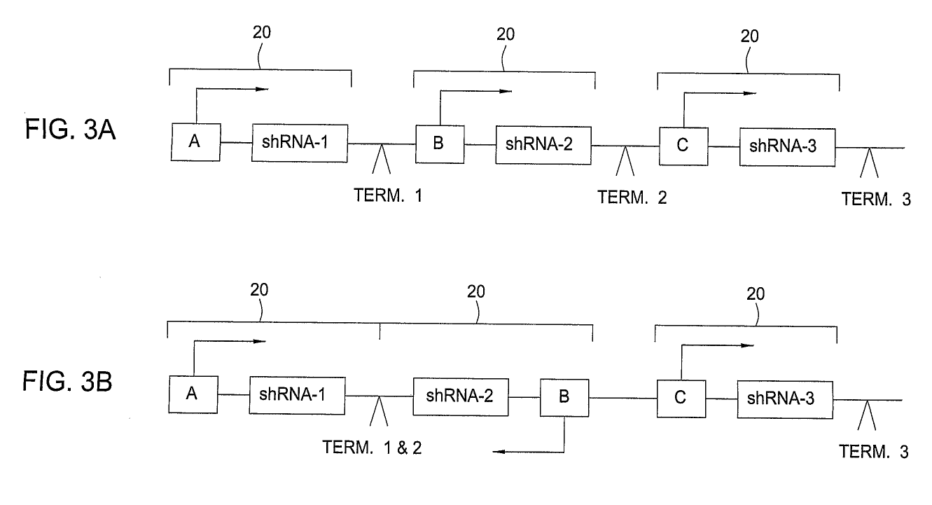

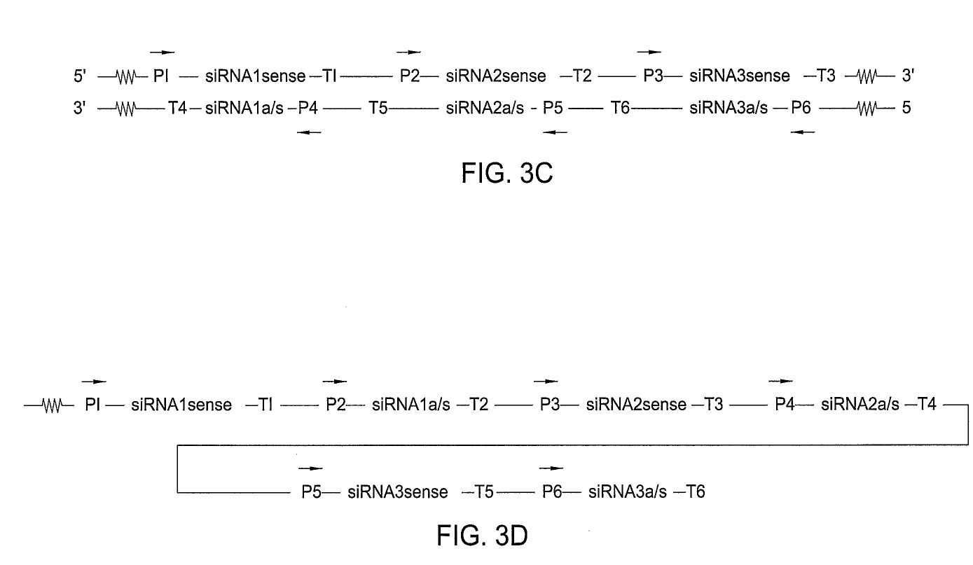

FIGS. 3A and 3B show multiple-promoter RNAi expression constructs comprising alternative embodiments of multiple-promoter RNAi expression cassettes that express short shRNAs. shRNAs are short duplexes where the sense and antisense strands are linked by a hairpin loop. Once expressed, shRNAs are processed into RNAi agents. A, B and C represent three different promoter elements, and the arrows indicate the direction of transcription. Term1, Term2, and Term3 represent three different termination sequences, and shRNA-1, shRNA-2 and shRNA-3 represent three different shRNA sequences. The multiple-promoter RNAi expression cassettes in both embodiments extend from the box marked A to the Term3. FIG. 3A shows each of the three promoter/RNAi/terminator components (20) in the same orientation within the cassette, while FIG. 3B shows the promoter/RNAi/terminator components for shRNA-1 and shRNA-3 in one orientation, and the promoter/RNAi/terminator component for sh-RNA2 in the opposite orientation (i.e., transcription takes place on both strands of the cassette). Other variations may be used as well.

FIGS. 3C and 3D show multiple-promoter RNAi expression constructs comprising alternative embodiments of multiple-promoter RNAi expression cassettes that express RNAi agents without a hairpin loop. In both figures, P1, P2, P3, P4, P5 and P6 represent promoter elements (with arrows indicating the direction of transcription); and T1, T2, T3, T4, T5, and T6 represent termination elements. Also in both figures, RNAi1 sense and RNAi1 antisense (a/s) are complements, RNAi2 sense and RNAi2 a/s are complements, and RNAi3 sense and RNAi3 a/s are complements.

In the embodiment shown in FIG. 3C, all three RNAi sense sequences are transcribed from one strand (via P1, P2 and P3), while the three RNAi a/s sequences are transcribed from the complementary strand (via P4, P5, P6). In this particular embodiment, the termination element of RNAi1a/s (T4) falls between promoter P1 and the RNAi 1 sense sequence; while the termination element of RNAi1 sense (T1) falls between the RNAi 1a/s sequence and its promoter, P4. This motif is repeated such that if the top strand shown in FIG. 3C is designated the (+) strand and the bottom strand is designated the (−) strand, the elements encountered moving from left to right would be P1(+), T4(−), RNAi1 (sense and a/s), T1(+), P4(−), P2(+), T5(−), RNAi2 (sense and a/s), T2(+), P5(−), P3(+), T6(−), RNAi3 (sense and a/s), T3(+), and P6(−). Various alternatives on this generalized construct are possible, wherein antisense sequences can be placed on the “top” strand, all such embodiments are envisioned by the present invention.

In an alternative embodiment shown in FIG. 3D, all RNAi sense and antisense sequences are transcribed from the same strand. One skilled in the art appreciates that any of the embodiments of the multiple-promoter RNAi expression cassettes shown in FIGS. 3A through 3D may be used for certain applications, as well as combinations or variations thereof.

In some embodiments, promoters of variable strength may be employed. For example, use of two or more strong promoters (such as a Pol III-type promoter) may tax the cell, by, e.g., depleting the pool of available nucleotides or other cellular components needed for transcription. In addition or alternatively, use of several strong promoters may cause a toxic level of expression of RNAi agents in the cell. Thus, in some embodiments one or more of the promoters in the multiple-promoter RNAi expression cassette may be weaker than other promoters in the cassette, or all promoters in the cassette may express RNAi agents at less than a maximum rate. Promoters also may or may not be modified using molecular techniques, or otherwise, e.g., through regulation elements, to attain weaker levels of transcription.

Promoters useful in some embodiments of the present invention may be tissue-specific or cell-specific. The term “tissue specific” as it applies to a promoter refers to a promoter that is capable of directing selective expression of a nucleotide sequence of interest to a specific type of tissue (e.g., stem cells) in the relative absence of expression of the same nucleotide sequence of interest in a different type of tissue (e.g., muscle). The term “cell-specific” as applied to a promoter refers to a promoter which is capable of directing selective expression of a nucleotide sequence of interest in a specific type of cell in the relative absence of expression of the same nucleotide sequence of interest in a different type of cell within the same tissue (see, e.g., Higashibata, et al., J. Bone Miner. Res. January 19 (1):78-88 (2004); Hoggatt, et al., Circ. Res., December 91(12):1151-59 (2002); Sohal, et al., Circ. Res. July 89(1):20-25 (2001); and Zhang, et al., Genome Res. January 14(1):79-89 (2004)). The term “cell-specific” when applied to a promoter also means a promoter capable of promoting selective expression of a nucleotide sequence of interest in a region within a single tissue. Alternatively, promoters may be constitutive or regulatable. Additionally, promoters may be modified so as to possess different specificities.

The term “constitutive” when made in reference to a promoter means that the promoter is capable of directing transcription of an operably linked nucleic acid sequence in the absence of a specific stimulus (e.g., heat shock, chemicals, light, etc.). Typically, constitutive promoters are capable of directing expression of a coding sequence in substantially any cell and any tissue. The promoters used to transcribe the RNAi agents preferably are constitutive promoters, such as the promoters for ubiquitin, CMV, β-actin, histone H4, EF-1alfa or pgk genes controlled by RNA polymerase II, or promoter elements controlled by RNA polymerase I. In other embodiments, a Pol II promoter such as CMV, SV40, U1, β-actin or a hybrid Pol II promoter is employed. In other embodiments, promoter elements controlled by RNA polymerase III are used, such as the U6 promoters (U6-1, U6-8, U6-9, e.g.), H1 promoter, 7SL promoter, the human Y promoters (hY1, hY3, hY4 (see Maraia, et al., Nucleic Acids Res 22(15): 3045-52 (1994)) and hY5 (see Maraia, et al., Nucleic Acids Res. 24(18): 3552-59 (1994)), the human MRP-7-2 promoter, Adenovirus VAl promoter, human tRNA promoters, the 5s ribosomal RNA promoters, as well as functional hybrids and combinations of any of these promoters.

Alternatively in some embodiments it may be optimal to select promoters that allow for inducible expression of the RNAi agent. A number of systems for inducible expression using such promoters are known in the art, including but not limited to the tetracycline responsive system and the lac operator-repressor system (see WO 03/022052 A1; and US 2002/0162126 A1), the ecdysone regulated system, or promoters regulated by glucocorticoids, progestins, estrogen, RU-486, steroids, thyroid hormones, cyclic AMP, cytokines, the calciferol family of regulators, or the metallothionein promoter (regulated by inorganic metals).

One or more enhancers also may be present in the viral multiple-promoter RNAi expression construct to increase expression of the gene of interest. Enhancers appropriate for use in embodiments of the present invention include the Apo E HCR enhancer, the CMV enhancer that has been described recently (see, Xia et al, Nucleic Acids Res. 31-17 (2003)), and other enhancers known to those skilled in the art.

The RNAi sequences encoded by the RNAi expression cassettes of the present invention result in the expression of small interfering RNAs that are short, double-stranded RNAs that are not toxic in normal mammalian cells. There is no particular limitation in the length of the ddRNAi agents of the present invention as long as they do not show cellular toxicity. RNAis can be, for example, 15 to 49 bp in length, preferably 15 to 35 bp in length, and are more preferably 19 to 29 bp in length. The double-stranded RNA portions of RNAis may be completely homologous, or may contain non-paired portions due to sequence mismatch (the corresponding nucleotides on each strand are not complementary), bulge (lack of a corresponding complementary nucleotide on one strand), and the like. Such non-paired portions can be tolerated to the extent that they do not significantly interfere with RNAi duplex formation or efficacy.

The termini of a ddRNAi agent according to the present invention may be blunt or cohesive (overhanging) as long as the ddRNAi agent effectively silences the target gene. The cohesive (overhanging) end structure is not limited only to a 3′ overhang, but a 5′ overhanging structure may be included as long as the resulting ddRNAi agent is capable of inducing the RNAi effect. In addition, the number of overhanging nucleotides may be any number as long as the resulting ddRNAi agent is capable of inducing the RNAi effect. For example, if present, the overhang may consist of 1 to 8 nucleotides; preferably it consists of 2 to 4 nucleotides.

The ddRNAi agent utilized in the present invention may have a stem-loop structured precursor (shRNA) in which the ends of the double-stranded RNA are connected by a single-stranded, linker RNA. The length of the loop portion of the shRNA may be 5 to 20 bp in length, and is preferably 5 to 9 bp in length.

The nucleic acid sequences that are targets for the RNAi expression cassettes of the present invention include genes that are involved in apoptosis or differentiation in general, including but not limited to telomere loss. The sequences for the RNAi agent or agents are selected based upon the genetic sequence of the target gene sequence(s); and preferably are based on regions of the target gene sequences that are conserved. Methods of alignment of sequences for comparison and RNAi sequence selection are well known in the art. The determination of percent identity between two or more sequences can be accomplished using a mathematical algorithm. Preferred, non-limiting examples of such mathematical algorithms are the algorithm of Myers and Miller (1988); the search-for-similarity-method of Pearson and Lipman (1988); and that of Karlin and Altschul (1993). Preferably, computer implementations of these mathematical algorithms are utilized. Such implementations include, but are not limited to: CLUSTAL in the PC/Gene program (available from Intelligenetics, Mountain View, Calif.); the ALIGN program (Version 2.0), GAP, BESTFIT, BLAST, FASTA, Megalign (using Jotun Hein, Martinez, Needleman-Wunsch algorithms), DNAStar Lasergene (see www.dnastar.com) and TFASTA in the Wisconsin Genetics Software Package, Version 8 (available from Genetics Computer Group (GCG), 575 Science Drive, Madison, Wis., USA). Alignments using these programs can be performed using the default parameters or parameters selected by the operator. The CLUSTAL program is well described by Higgins. The ALIGN program is based on the algorithm of Myers and Miller; and the BLAST programs are based on the algorithm of Karlin and Altschul. Software for performing BLAST analyses is publicly available through the National Center for Biotechnology Information (http://www.ncbi.nlm.nih.gov/).

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Typically, inhibition of target sequences by RNAi requires a high degree of sequence homology between the target sequence and the sense strand of the RNAi molecules. In some embodiments, such homology is higher than about 70%, and may be higher than about 75%. Preferably, homology is higher than about 80%, and is higher than 85% or even 90%. More preferably, sequence homology between the target sequence and the sense strand of the RNAi is higher than about 90%, 91%, 92%, 93%, 94%, 95%, 96%, 97%, 98% or 99%.

In addition to selecting the RNAi sequences based on conserved regions of a target gene, selection of the RNAi sequences may be based on other factors. Despite a number of attempts to devise selection criteria for identifying sequences that will be effective in RNAi based on features of the desired target sequence (e.g., percent GC content, position from the translation start codon, or sequence similarities based on an in silico sequence database search for homologs of the proposed RNAi, thermodynamic pairing criteria), it is presently not possible to predict with much degree of confidence which of the myriad possible candidate RNAi sequences corresponding to a target gene, in fact, elicit an optimal RNA silencing response. Instead, individual specific candidate RNAi polynucleotide sequences typically are generated and tested to determine whether interference with expression of a desired target can be elicited.

As stated, the ddRNAi agent coding regions of RNAi expression cassette are operatively linked to terminator elements. In one embodiment, the terminators comprise stretches of four or more thymidine residues. In embodiments where multiple promoter cassettes are used, the terminator elements used all may be different and are matched to the promoter elements from the gene from which the terminator is derived. Such terminators include the SV40 poly A, the Ad VA1 gene, the 5S ribosomal RNA gene, and the terminators for human t-RNAs. In addition, promoters and terminators may be mixed and matched, as is commonly done with RNA pol II promoters and terminators.

In addition, the RNAi expression cassettes may be configured where multiple cloning sites and/or unique restriction sites are located strategically, such that the promoter, ddRNAi agents and terminator elements are easily removed or replaced. The RNAi expression cassettes may be assembled from smaller oligonucleotide components using strategically located restriction sites and/or complementary sticky ends. The base vector for one approach according to embodiments of the present invention consists of plasmids with a multilinker in which all sites are unique (though this is not an absolute requirement). Sequentially, each promoter is inserted between its designated unique sites resulting in a base cassette with one or more promoters, all of which can have variable orientation. Sequentially, again, annealed primer pairs are inserted into the unique sites downstream of each of the individual promoters, resulting in a single-, double- or multiple-expression cassette construct. The insert can be moved into, e.g. an AAV backbone using two unique enzyme sites (the same or different ones) that flank the single-, double- or multiple-expression cassette insert.

When using a ddRNAi agent, the RNAi expression cassette is ligated into a delivery vector. The constructs into which the RNAi expression cassette is inserted and used for high efficiency transduction and expression of the ddRNAi agents in various cell types may be derived from viruses and are compatible with viral delivery; alternatively, non-viral delivery method may be used. Generation of the construct can be accomplished using any suitable genetic engineering techniques well known in the art, including without limitation, the standard techniques of PCR, oligonucleotide synthesis, restriction endonuclease digestion, ligation, transformation, plasmid purification, and DNA sequencing. If the construct is a viral construct, the construct preferably comprises, for example, sequences necessary to package the RNAi expression construct into viral particles and/or sequences that allow integration of the RNAi expression construct into the target cell genome. The viral construct also may contain genes that allow for replication and propagation of virus, though in other embodiments such genes will be supplied in trans. Additionally, the viral construct may contain genes or genetic sequences from the genome of any known organism incorporated in native form or modified. For example, a preferred viral construct may comprise sequences useful for replication of the construct in bacteria.

The construct also may contain additional genetic elements. The types of elements that may be included in the construct are not limited in any way and may be chosen by one with skill in the art. For example, additional genetic elements may include a reporter gene, such as one or more genes for a fluorescent marker protein such as GFP or RFP; an easily assayed enzyme such as beta-galactosidase, luciferase, beta-glucuronidase, chloramphenical acetyl transferase or secreted embryonic alkaline phosphatase; or proteins for which immunoassays are readily available such as hormones or cytokines. Other genetic elements that may find use in embodiments of the present invention include those coding for proteins which confer a selective growth advantage on cells such as adenosine deaminase, aminoglycodic phosphotransferase, dihydrofolate reductase, hygromycin-B-phosphotransferase, drug resistance, or those genes coding for proteins that provide a biosynthetic capability missing from an auxotroph. If a reporter gene is included along with the RNAi expression cassette, an internal ribosomal entry site (IRES) sequence can be included. Preferably, the additional genetic elements are operably linked with and controlled by an independent promoter/enhancer. In addition a suitable origin of replication for propagation of the construct in bacteria may be employed. The sequence of the origin of replication generally is separated from the ddRNAi agent and other genetic sequences that are to be expressed in the cells. Such origins of replication are known in the art and include the pUC, ColE1, 2-micron or SV40 origins of replication.

A viral delivery system based on any appropriate virus may be used to deliver the RNAi expression constructs of the present invention. In addition, hybrid viral systems may be of use. The choice of viral delivery system will depend on various parameters, such as efficiency of delivery into cells, transduction efficiency of the system, pathogenicity, immunological and toxicity concerns, and the like. It is clear that there is no single viral system that is suitable for all applications. When selecting a viral delivery system to use in the present invention, it is important to choose a system where RNAi expression construct-containing viral particles are preferably: 1) reproducibly and stably propagated; 2) able to be purified to high titers; and 3) able to mediate targeted delivery (delivery of the multiple-promoter RNAi expression construct to the desired cells without widespread dissemination).

In general, the five most commonly used classes of viral systems used in gene therapy can be categorized into two groups according to whether their genomes integrate into host cellular chromatin (oncoretroviruses and lentiviruses) or persist in the cell nucleus predominantly as extrachromosomal episomes (adeno-associated virus, adenoviruses and herpesviruses).

For example, in one embodiment of the present invention, viruses from the Parvoviridae family are utilized. The Parvoviridae is a family of small single-stranded, non-enveloped DNA viruses with genomes approximately 5000 nucleotides long. Included among the family members is adeno-associated virus (AAV), a dependent parvovirus that by definition requires co-infection with another virus (typically an adenovirus or herpesvirus) to initiate and sustain a productive infectious cycle. In the absence of such a helper virus, AAV is still competent to infect or transduce a target cell by receptor-mediated binding and internalization, penetrating the nucleus in both non-dividing and dividing cells.

Once in the nucleus, the virus uncoats and the transgene is expressed from a number of different forms—the most persistent of which are circular monomers. AAV will integrate into the genome of 1-5% of cells that are stably transduced (Nakai, et al., J. Virol. 76:11343-349 (2002)). Expression of the transgene can be exceptionally stable and in one study with AAV delivery of Factor IX, a dog model continues to express therapeutic levels of the protein 5.0 years after a single direct infusion with the virus. Because progeny virus is not produced from AAV infection in the absence of helper virus, the extent of transduction is restricted only to the initial cells that are infected with the virus. It is this feature that makes AAV a preferred gene therapy vector for the present invention. Furthermore, unlike retrovirus, adenovirus, and herpes simplex virus, AAV appears to lack human pathogenicity and toxicity (Kay, et al., Nature. 424: 251 (2003) and Thomas, et al., Nature Reviews, Genetics 4:346-58 (2003)).

Typically, the genome of AAV contains only two genes. The “rep” gene codes for at least four separate proteins utilized in DNA replication. The “cap” gene product is spliced differentially to generate the three proteins that comprise the capsid of the virus. When packaging the genome into nascent virus, only the Inverted Terminal Repeats (ITRs) are obligate sequences; rep and cap can be deleted from the genome and be replaced with heterologous sequences of choice. However, in order to produce the proteins needed to replicate and package the AAV-based heterologous construct into nascent virion, the rep and cap proteins must be provided in trans. The helper functions normally provided by co-infection with the helper virus, such as adenovirus or herpesvirus mentioned above also can be provided in trans in the form of one or more DNA expression plasmids. Since the genome normally encodes only two genes it is not surprising that, as a delivery vehicle, AAV is limited by a packaging capacity of 4.5 single stranded kilobases (kb). However, although this size restriction may limit the genes that can be delivered for replacement gene therapies, it does not adversely affect the packaging and expression of shorter sequences such as RNAi.

The utility of AAV for RNAi applications was demonstrated in experiments where AAV was used to deliver shRNA in vitro to inhibit p53 and Caspase 8 expression (Tomar et al., Oncogene. 22: 5712-15 (2003)). Following cloning of the appropriate sequences into a gutted AAV-2 vector, infectious AAV virions were generated in HEK293 cells and used to infect HeLa S3 cells. A dose-dependent decrease of endogenous Caspase 8 and p53 levels was demonstrated. Boden et al. also used AAV to deliver shRNA in vitro to inhibit HIV replication in tissue culture systems (Boden, et al., J. Virol. 77(21): 115231-35 (2003)) as assessed by p24 production in the spent media.

However, technical hurdles must be addressed when using AAV as a vehicle for RNAi expression constructs. For example, various percentages of the human population may possess neutralizing antibodies against certain AAV serotypes. However, since there are several AAV serotypes—some of which the percentage of individuals harboring neutralizing antibodies is very low—other serotypes can be used or pseudo-typing may be employed. There are at least eight different serotypes that have been characterized, with dozens of others, which have been isolated but have been less well described. Another limitation is that as a result of a possible immune response to AAV, AAV-based therapy may only be administered once; however, use of alternate, non-human derived serotypes may allow for repeat administrations. Administration route, serotype, and composition of the delivered genome all influence tissue specificity.

Another limitation in using unmodified AAV systems with the RNAi expression constructs is that transduction can be inefficient. Stable transduction in vivo may be limited to 5-10% of cells. However, different methods are known in the art to boost stable transduction levels. One approach is utilizing pseudotyping, where AAV-2 genomes are packaged using cap proteins derived from other serotypes. For example, by substituting the AAV-5 cap gene for its AAV-2 counterpart, Mingozzi et al. increased stable transduction to approximately 15% of hepatocytes (Mingozzi, et al., J. Virol. 76(20): 10497-502 (2002)). Thomas et al., transduced over 30% of mouse hepatocytes in vivo using the AAV8 capsid gene (Thomas, et al., J. Virol. in press). Grimm et al. (Blood. 2003-02-0495) exhaustively pseudotyped AAV-2 with AAV-1, AAV-3B, AAV-4, AAV-5, and AAV-6 for tissue culture studies. The highest levels of transgene expression were induced by virion which had been pseudotyped with AAV-6; producing nearly 2000% higher transgene expression than AAV-2. Thus, the present invention contemplates use of a pseudotyped AAV virus to achieve high transduction levels, with a corresponding increase in the expression of the RNAi multiple-promoter expression constructs.

Another viral delivery system useful with the RNAi expression constructs of the present invention is a system based on viruses from the family Retroviridae. Retroviruses comprise single-stranded RNA animal viruses that are characterized by two unique features. First, the genome of a retrovirus is diploid, consisting of two copies of the RNA. Second, this RNA is transcribed by the virion-associated enzyme reverse transcriptase into double-stranded DNA. This double-stranded DNA or provirus can then integrate into the host genome and be passed from parent cell to progeny cells as a stably-integrated component of the host genome.

In some embodiments, lentiviruses are the preferred members of the retrovirus family for use in the present invention. Lentivirus vectors are often pseudotyped with vesicular stomatitis virus glycoprotein (VSV-G), and have been derived from the human immunodeficiency virus (HIV), the etiologic agent of the human acquired immunodeficiency syndrome (AIDS); visan-maedi, which causes encephalitis (visna) or pneumonia in sheep; equine infectious anemia virus (EIAV), which causes autoimmune hemolytic anemia and encephalopathy in horses; feline immunodeficiency virus (FIV), which causes immune deficiency in cats; bovine immunodeficiency virus (BIV) which causes lymphadenopathy and lymphocytosis in cattle; and simian immunodeficiency virus (SIV), which causes immune deficiency and encephalopathy in non-human primates. Vectors that are based on HIV generally retain <5% of the parental genome, and <25% of the genome is incorporated into packaging constructs, which minimizes the possibility of the generation of reverting replication-competent HIV. Biosafety has been further increased by the development of self-inactivating vectors that contain deletions of the regulatory elements in the downstream long-terminal-repeat sequence, eliminating transcription from the integrated provirus.

Reverse transcription of the retroviral RNA genome occurs in the cytoplasm. Unlike C-type retroviruses, the lentiviral cDNA complexed with other viral factors—known as the pre-initiation complex—is able to translocate across the nuclear membrane and transduce non-dividing cells. A structural feature of the viral cDNA-a DNA flap-seems to contribute to efficient nuclear import. This flap is dependent on the integrity of a central polypurine tract (cPPT) that is located in the viral polymerase gene, so most lentiviral-derived vectors retain this sequence. Lentiviruses have broad tropism, low inflammatory potential, and result in an integrated vector. The main limitations are that integration might induce oncogenesis in some applications. The main advantage to the use of lentiviral vectors is that gene transfer is persistent in most tissues or cell types due to integration of the recombinant genome.

A lentiviral-based construct used to express the ddRNAi agents preferably comprises sequences from the 5′ and 3′ LTRs of a lentivirus. More preferably the viral construct comprises an inactivated or self-inactivating 3′ LTR from a lentivirus. The 3′ LTR may be made self-inactivating by any method known in the art. In a preferred embodiment, the U3 element of the 3′ LTR contains a deletion of its enhancer sequence, preferably the TATA box, Sp1 and NF-kappa B sites. As a result of the self-inactivating 3′ LTR, the provirus that is integrated into the host cell genome will comprise an inactivated 5′ LTR. The LTR sequences may be LTR sequences from any lentivirus from any species. The lentiviral-based construct also may incorporate sequences for MMLV or MSCV, RSV or mammalian genes. In addition, the U3 sequence from the lentiviral 5′ LTR may be replaced with a promoter sequence in the viral construct. This may increase the titer of virus recovered from the packaging cell line. An enhancer sequence may also be included.

Other viral or non-viral systems known to those skilled in the art may be used to deliver the RNAi expression cassettes of the present invention to cells, including but not limited to gene-deleted adenovirus-transposon vectors that stably maintain virus-encoded transgenes in vivo through integration into host cells (see Yant, et al., Nature Biotech. 20:999-1004 (2002)); systems derived from Sindbis virus or Semliki forest virus (see Perri, et al, J. Virol. 74(20):9802-07 (2002)); systems derived from Newcastle disease virus or Sendai virus; or mini-circle DNA vectors devoid of bacterial DNA sequences (see Chen, et al., Molecular Therapy. 8(3):495-500 (2003)). Mini-circle DNA as described in U.S. Publ. No. 2004/0214329 discloses vectors that provide for persistently high levels of protein. The circular vectors are characterized by being devoid of expression-silencing bacterial sequences, and may include a unidirectional site-specific recombination product sequence in addition to an expression cassette.

In addition, hybrid viral systems may be used to combine useful properties of two or more viral systems. For example, the site-specific integration machinery of wild-type AAV may be coupled with the efficient internalization and nuclear targeting properties of adenovirus. AAV in the presence of adenovirus or herpesvirus undergoes a productive replication cycle; however, in the absence of helper functions, the AAV genome integrates into a specific site on chromosome 19. Integration of the AAV genome requires expression of the AAV rep protein. As conventional rAAV vectors are deleted for all viral genes including rep, they are not able to specifically integrate into chromosome 19. However, this feature may be exploited in an appropriate hybrid system. In addition, non-viral genetic elements may be used to achieve desired properties in a viral delivery system, such as genetic elements that allow for site-specific recombination.

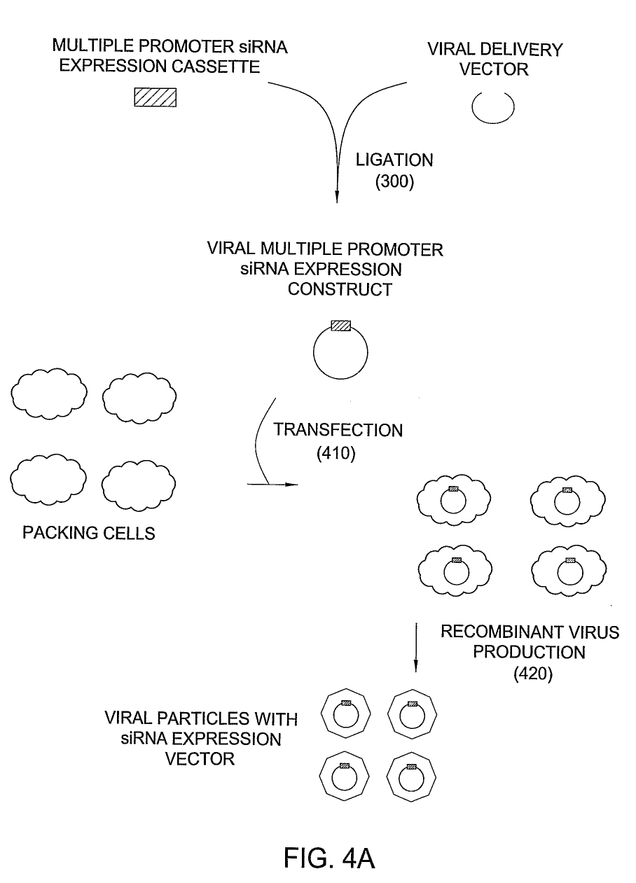

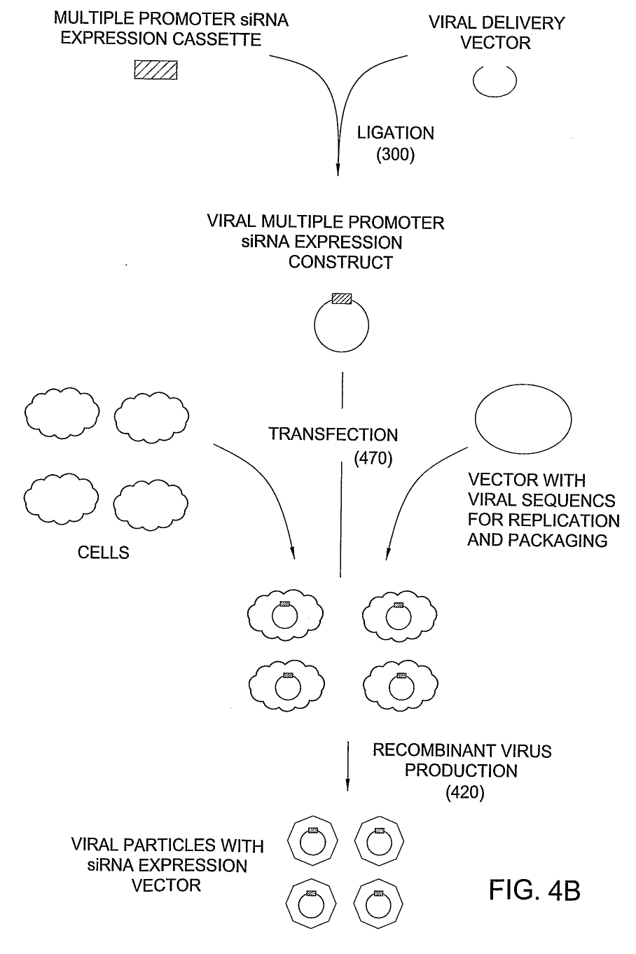

In step 400 of FIG. 1A, the RNAi expression construct is packaged into viral particles. Any method known in the art may be used to produce infectious viral particles whose genome comprises a copy of the viral RNAi expression construct. FIGS. 4A and 4B show alternative methods for packaging the RNAi expression constructs of the present invention into viral particles for delivery. The method in FIG. 4A utilizes packaging cells that stably express in trans the viral proteins that are required for the incorporation of the viral RNAi expression construct into viral particles, as well as other sequences necessary or preferred for a particular viral delivery system (for example, sequences needed for replication, structural proteins and viral assembly) and either viral-derived or artificial ligands for tissue entry. In FIG. 4A, an RNAi expression cassette is ligated to a viral delivery vector (step 300), and the resulting viral RNAi expression construct is used to transfect packaging cells (step 410). The packaging cells then replicate viral sequences, express viral proteins and package the viral RNAi expression constructs into infectious viral particles (step 420). The packaging cell line may be any cell line that is capable of expressing viral proteins, including but not limited to 293, HeLa, A549, PerC6, D17, MDCK, BHK, bing cherry, phoenix, Cf2Th, or any other line known to or developed by those skilled in the art. One packaging cell line is described, for example, in U.S. Pat. No. 6,218,181.

Alternatively, a cell line that does not stably express necessary viral proteins may be co-transfected with two or more constructs to achieve efficient production of functional particles. One of the constructs comprises the viral RNAi expression construct, and the other plasmid(s) comprises nucleic acids encoding the proteins necessary to allow the cells to produce functional virus (replication and packaging construct) as well as other helper functions. The method shown in FIG. 4B utilizes cells for packaging that do not stably express viral replication and packaging genes. In this case, the RNAi expression construct is ligated to the viral delivery vector (step 300) and then co-transfected (step 470) with one or more vectors that express the viral sequences necessary for replication and production of infectious viral particles. The cells replicate viral sequences, express viral proteins and package the viral RNAi expression constructs into infectious viral particles (step 420).

The packaging cell line or replication and packaging construct may not express envelope gene products. In these embodiments, the gene encoding the envelope gene can be provided on a separate construct that is co-transfected with the viral RNAi expression construct. As the envelope protein is responsible, in part, for the host range of the viral particles, the viruses may be pseudotyped. As described supra, a “pseudotyped” virus is a viral particle having an envelope protein that is from a virus other than the virus from which the genome is derived. One with skill in the art can choose an appropriate pseudotype for the viral delivery system used and cell to be targeted. In addition to conferring a specific host range, a chosen pseudotype may permit the virus to be concentrated to a very high titer. Viruses alternatively can be pseudotyped with ecotropic envelope proteins that limit infection to a specific species (e.g., ecotropic envelopes allow infection of, e.g., murine cells only, where amphotropic envelopes allow infection of, e.g., both human and murine cells.) In addition, genetically-modified ligands can be used for cell-specific targeting, such as the asialoglycoprotein for hepatocytes, or transferrin for receptor-mediated binding.

After production in a packaging cell line, the viral particles containing the RNAi expression cassettes are purified and quantified (titered). Purification strategies include density gradient centrifugation, or, preferably, column chromatographic methods.

Multiple-promoter RNAi expression cassettes used in certain embodiments of the present invention are particularly useful in promoting stem cell expansion and preventing differentiation because RNAi agents against multiple genes involved these processes can be targeted simultaneously. For example, one or more genes that repress telomerase and/or cytokines can be repressed at the same time.

Alternatively, the RNAi expression cassettes may be delivered into cells by non-viral means, such as bacterial vectors or mini-circles (see Chen, et al., Molecular Therapy. 8(3):495-500 (2003) and US Pat. Pub. 2004/0214329, incorporated herein by reference). Mini-circles are non-viral DNA vectors that provide for persistently high expression of nucleic acid transcription. Mini-circle vectors are characterized by being devoid of expression-silencing bacterial DNA sequences, and may include a unidirectional site-specific recombination product sequence in addition to a ddRNAi expression cassette.

The siRNAs and ddRNAi expression cassettes of the present invention may be delivered into cells in vitro or ex vivo then placed into an animal to effect therapy. A variety of techniques are available and well known for delivery of nucleic acids into cells, for example, liposome- or micelle-mediated transfection or transformation, or by cell mating or by microinjection or other techniques known in the art.

RNAi Agents

Any RNAi agent that is capable of retarding or arresting apoptosis or differentiation while promoting expansion is appropriate for incorporation into stem cells of the present invention. Stem cells include but are not limited to hematopoietic stem cells, myoblasts, osteblasts, neural stem cells and embryonic stem cells. One important feature of this invention is that the cell could be used for delivering a therapeutic transgene and would retain the therapeutic transgene even as it proliferates or differentiates into specialized cells. Most of the cell-based gene therapies attempted so far have used viral vehicles to introduce the transgene into the hematopoietic stem cell. One way to accomplish this is to insert the therapeutic transgene into the one of the chromosomes of the stem cell. Retroviruses are able to do this, and for this reason, they are often used as the vehicle for infecting the stem cell and introducing the therapeutic transgene into the chromosomal DNA. Many retroviruses are only efficient at infecting cells that are actively dividing. RNAi agents of this invention would preferably promote cell growth while retaining any transgene inserted for therapeutic purposes. This would greatly increase the number of stem cells that actually receive the therapeutic transgene. Insertion of an RNAi agent that would promote cell division, while retarding differentiation, would be beneficial for both research and therapeutic purposes.

The process of differentiation of stem cells starts when a stem cell generates a progenitor cell. Progenitor or precursor cells in fetal and adult tissues are partly differentiated cells that divide and give rise to differentiated cells. Such cells are usually regarded as committed to differentiating along a particular cellular development pathway. Stem cells harvested for the purpose of gene therapy can be collected either from bone marrow or from peripheral blood using a method known as aphaeresis. Stem cells collected in either manner can then be genetically modified by the process of this invention and used for further study or genetically modified to create a stem cell with a desired phenotype.

The RNAi agents according to the present invention include those that can act upon pathways that direct the self renewing capacity of stem cells. These include, but are not limited to signaling and telomere maintenance pathways involving proteins such as Notch, WNT, HOXB4, and TERT. For review see Elwood, Cancer Control 11 (2): 77-85 (2004).

Notch signaling is involved in the regulation of many cell fate determination events in both embryonic development and adult tissue homeostasis. Notch1 and Notch2 molecules inhibit myeloid differentiation in a cytokine-specific manner where the Notch cytokine response domain is necessary for this functional specificity. The phosphorylation of Notch proteins is also critical to the activity of Notch in response to cytokine signals (Ingles-Esteve, et al., J. Biol. Chem. 276(48): 44873-44880 (2001). Phosphorylation of Notch proteins is controlled by granulocyte colony-stimulating factor (G-CSF). Inhibition of G-CSF protein leads to prevention of phosphorylation of Notch proteins and the consequent delay of the differentiation pathway.

Another protein, HOXB4 has been demonstrated to regulate HSCs. Overexpression of HOXB4 can provide an effective method for self-renewal of stem cells. Retroviral overexpression of HOXB4 for 10 to 14 days in vitro could increase the number of repopulating HSCs by 40-fold compared with fresh bone marrow stem cells (Antonchuk, et. al., Exp. Hematol. 29(9):1125-34 (2001).

Further, it has been shown that HOXB4 protein can stimulate self-renewal of HSCs in culture. The level of HOXB4 expression can be increased by the activation of WNT.

WNT proteins stimulate the survival/proliferation of hematopoietic progenitors, demonstrating that WNT proteins comprise a class of hematopoietic cell regulators. WNT is repressed by TCF1 (Austin, et al., Blood 89(10): 3624-2625 (1997)). The inhibition of TCF1 by RNAi agents therefore will have the affect of expanding stem cell populations in vitro or in vivo, while inhibiting differentiation.

The signal transducer Stat5 plays a key role in the regulation of hematopoietic differentiation and hematopoietic stem cell function. Induction of Stat5 at the point of origin of the hematopoietic lineage (from day 4 to day 6 of embryoid body differentiation) significantly enhances the number of hematopoietic progenitors with colony-forming potential (Kyba, PNAS 100:11904-11910 (2003)). It does so without significantly altering the total number or inducing apoptosis, suggesting a cell-intrinsic effect of Stat5 on either the developmental potential or clonogenicity of the hematopoietic cell population.

Another factor, SMRT (silencing mediator for retinoic acid receptor and thyroid hormone receptor) strongly represses STAT5-dependent transcription in vitro (Nakajima, Embo 20: 6836-6844 (2001)). The inhibition of SMRT by RNAi agents affects the expansion of stem cell populations in vitro or in vivo, while inhibiting differentiation.

The present invention is predicated in part on the use of genetic agents which facilitate silencing of one or more transcriptionally active genetic regions via RNAi wherein those transcriptionally active genetic regions are directly or indirectly associated with the stem cell differentiation and/or apoptosis. Such transcriptionally active regions are also referred to herein as “stem cell associated genetic targets” or “SCATs”. ddRNAi-mediated silencing of one or more SCATs can affect control of one or more of the onset of cell differentiation or apoptosis in the subject or cell culture.

As used herein, the terms “stem cell genetic target” or “SCAT” refers to any genetic sequence or transcript thereof which is directly or indirectly associated with control of stem cell growth and differentiation in a vertebrate animal, particularly mammalian animals and most particularly in primate or rodent animals. Accordingly, a SCAT may be a gene directly associated with stem cell proliferation or a transcript thereof, a nucleic acid region which encodes a regulatory RNA, which is associated with stem cells, or the SCAT may comprise a protein-encoding or regulatory RNA-encoding nucleic acid sequence which itself may not be associated with stem cells, but the expression of which may modulate the expression of a gene or regulatory RNA which is directly associated with stem cells. Accordingly, the term SCAT should be understood to include genetic targets which are directly or indirectly involved in the onset of differentiation or apoptosis in a subject or cell culture.

The present invention is predicated in part on the use of RNAi to silence the expression of one or more SCATs, which in turn controls the onset, of differentiation or apoptosis of HSCs in a subject or cell culture. Table 1 shows a list of exemplary SCAT sequences. The term “silencing of expression” in this context includes regulating the amount of functional RNA transcript derived from the SCAT. Regulating the amount of functional RNA transcript may occur by facilitating transcript degradation or facilitating formation of nucleic acid based molecules which inhibit translation. The genetic RNAi agents of the present invention promote or facilitate post-transcriptional gene silencing. As used herein “functional RNA transcript” refers to an RNA transcript which is able to perform its usual function. For example, in the case of the SCAT being a protein-encoding gene, a “functional RNA transcript” would be a translatable mRNA. However, in the case where a SCAT encodes a non-translated regulatory RNA, a “functional RNA transcript” would be an RNA transcript capable of effecting regulation of another genetic sequence.

TABLE 1
Exemplary SCAT sequences which may be targeted using ddRNAi

| | Entrez |
|---|---|
| | Gene |
| SCAT | ID No. |
| WNT2 wingless-type MMTV integration site family member | 7472 |
| 2 [Homo sapiens] | |
| Wnt2 wingless-related MMTV integration site 2 [Mus | 22413 |
| musculus] | |
| Wnt4 wingless-related MMTV integration site 4 [Mus | 22417 |
| musculus] | |
| WNT4 wingless-type MMTV integration site family, member | 54361 |
| 4 [Homo sapiens] | |
| WIF1 WNT inhibitory factor 1 [Homo sapiens] | 11197 |
| Wnt2 wingless-related MMTV integration site 2 [Rattus | 114487 |
| norvegicus] | |
| Wif1 Wnt inhibitory factor 1 [Mus musculus] | 24117 |
| Wif1 Wnt inhibitory factor 1 [Rattus norvegicus] | 114557 |
| Wnt3awingless-related MMTV integration site 3A [Mus | 22416 |
| musculus] | |
| WNT3A wingless-type MMTV integration site family, | 22416 |
| member 3A [Homo sapiens] | |
| Tcf1 transcription factor 1 [Rattus norvegicus] | 24817 |
| TCF1 transcription factor 1 [Homo sapiens] | 24817 |
| Tcf1 transcription factor 1 [Mus musculus] | 21405 |
| Tcf7 transcription factor 7, T-cell specific [Mus musculus] | 21414 |
| Tcf3 transcription factor 3 [Mus musculus] | 21415 |
| TCF3 transcription factor 3 (E2A immunoglobulin enhancer | 6929 |
| binding factors E12/E47) [Homo sapiens] | |
| TCF7L1 transcription factor 7-like 1 (T-ceIl specific, HMG- | 83439 |
| box) [Homo sapiens] | |
| TCF4 transcription factor 4 [Homo sapiens] | 6925 |
| Tcf4 transcription factor 4 [Mus musculus] | 21413 |
| Tcf4 transcription factor 4 [Rattus norvegicus] | 84382 |
| TPS3 tumor protein p53 (Li-Fraumeni syndrome) [Homo | 7157 |
| sapiens] | |
| Hoxb4 homeo box B4 [Mus musculus] | 15412 |
| HOXB4 homeo box B4 [Homo sapiens] | 3214 |
| HOXB3 homeo box B3 [Homo sapiens] | 3213 |
| Notch1 Notch gene homolog 1, (Drosophila) [Rattus | 25496 |
| norvegicus] | |
| NOTCH1 Notch homolog 1, translocation-associated | 4851 |
| (Drosophila) [Homo sapiens] | |
| Notch1 Notch gene homolog 1 (Drosophila) [Mus musculus] | 18128 |
| NOTCH2 Notch homolog 2 (Drosophila) [Homo sapiens] | 4853 |
| NOTCH3 Notch homolog 3 (Drosophila) [Homo sapiens] | 4854 |
| Notch2 Notch gene homolog 2 (Drosophila) [Mus musculus] | 18129 |
| Notch2 notch gene homolog 2, (Drosophila) [Rattus | 29492 |
| norvegicus] | |
| Notch3 Notch gene homolog 3 (Drosophila) [Mus musculus] | 18131 |
| NOTCH4 Notch homolog 4 (Drosophila) [Homo sapiens] | 4855 |
| Terf 1 telomeric repeat binding factor 1 [Mus musculus] | 21749 |
| TERF2 telomeric repeat binding factor 2 [Homo sapiens] | 7014 |
| Terf2 telomeric repeat binding factor 2 [Mus musculus] | 21750 |
| TERF1 telomeric repeat binding factor (NIMA-interacting) | 7013 |
| 1 [Homo sapiens] | |
| POT1 protection of telomeres 1 homolog (S. pombe) [Homo | 25913 |
| sapiens] | |
| Pot1 protection of telomeres 1 [Mus musculus] | 101185 |
| CSF3 colony stimulating factor 3 (granulocyte) [Homo sapiens | 1440 |
| Csf3 colony stimulating factor 3 (granulocyte) [Mus musculus] | 12985 |
| STAT3 signal transducer and activator of transcription 3 (acute- | 6774 |
| phase response factor) [Homo sapiens] | |
| CSF3R colony stimulating factor 3 receptor | 1441 |
| (granulocyte) [Homo sapiens] | |
| Csf3r colony stimulating factor 3 receptor (granulocyte) [Mus | 12986 |
| musculus] | |
| MYC v-myc myelocytomatosis viral oncogene homolog | 4609 |
| (avian) [Homo sapiens] | |
| TERT telomerase reverse transcriptase [Homo sapiens] | 7015 |
| MYCBP c-myc binding protein [Homo sapiens] | 26292 |
| MAD MAX dimerization protein 1 [Homo sapiens] | 4084 |
| Mad Max dimerization protein [Mus musculus] | 17119 |
| TERC telomerase RNA component [Homo sapiens] | 7012 |
| TEP1 telomerase-associated protein [Homo sapiens] | 7011 |
| TEP1 telomerase-associated protein [Mus musculus] | 21745 |
| USF1 upstream transcription factor 1 [Homo sapiens] | 7391 |
| USF1 upstream transcription factor 1 [Mus musculus] | 22278 |
| SMRT silencing mediator of retinoic acid and thyroid hormone | 9612 |
| receptors [Homo sapiens] | |
| SMRT silencing mediator of retinoic acid and thyroid hormone | 20602 |
| receptors [Mus musculus] | |

Other agents that are useful in conjunction with the present invention will be readily apparent to those of skill in the art.

Covalently Attached RNAi Agents

In some embodiments, the RNAi agent may be covalently bonded to a reactive group which may be associated with a carrier or coating for delivery. The art is replete with methods for preparing derivatized, polymerizable monomers, attaching nucleic acids onto polymeric surfaces and derivatizing nucleic acids and polymers to allow for this attachment (see, for example, Hermanson, Bioconjugate Techniques, Academic Press, 1996, and references therein). Common approaches include the use of coupling agents such as glutaraldehyde, cyanogen bromide, p-benzoquinone, succinic anhydrides, carbodiimides, diisocyanates, ethyl chloroformate, dipyridyl disulfide, epichlorohydrin, azides, among others, which serve as attachment vehicles for coupling reactive groups of derivatized nucleic acid molecules to reactive groups on a monomer or a polymer.

A polymer can be functionalized with reactive groups by, for example, including a moiety bearing a reactive group as an additive to a blend during manufacture of the polymer or polymer precursor. The additive is dispersed throughout the polymer matrix, but does not form an integral part of the polymeric backbone. In this embodiment, the surface of the polymeric material is altered or manipulated by the choice of additive or modifier characteristics. The reactive groups of the additive are used to bind the one or more RNAi agents to the polymer.

A useful method for preparing surface-functionalized polymeric materials by this method is set forth in, for example, U.S. Pat. No. 5,784,164 to Caldwell. In the Caldwell method, additives or modifiers are combined with the polymeric material during its manufacture. These additives or modifiers include compounds that have reactive sites, compounds that facilitate the controlled release of agents from the polymeric material into the surrounding environment, catalysts, compounds that promote adhesion between bioactive materials (such as an RNAi agent) and the polymeric material and compounds that alter the surface chemistry of the polymeric material. In another embodiment, polymerizable monomers bearing reactive groups are incorporated in the polymerization mixture. The functionalized monomers form part of the polymeric backbone and, preferably, present their reactive groups on the surface of the polymer.

Reactive groups contemplated in the practice of the present invention include functional groups, such as hydroxyl, carboxyl, carboxylic acid, amine groups, and the like, that promote physical and/or chemical interaction with the bioactive material. The particular compound employed as the modifier will depend on the chemical functionality of the biologically active RNAi agent and can readily be deduced by one of skill in the art. In the present embodiment, the reactive site binds a bioactive agent by covalent means. It will, however, be apparent to those of skill in the art that these reactive groups can also be used to adhere the RNAi agents to the polymer by hydrophobic/hydrophilic, ionic and other non-covalent mechanisms.

In addition to manipulating the composition and structure of the polymer during manufacture, a preferred polymer also can be modified using a surface derivitization technique. There are a number of surface-derivatization techniques appropriate for use in fabricating the RNAi agent/carrier and, ultimately, the therapeutic devices of the present invention. These techniques for creating functionalized polymeric surfaces (e.g., grafting techniques) are well known to those skilled in the art. For example, techniques based on ceric ion initiation, ozone exposure, corona discharge, UV irradiation and ionizing radiation (⁶⁰Co, X-rays, high energy electrons, plasma gas discharge) are known and can be used in the practice of the present invention.

Substantially any reactive group that can be reacted with a complementary component on an RNAi agent can be incorporated into a polymer and used to covalently attach the RNAi agent to the carrier coating of use in the invention. In a preferred embodiment, the reactive group is selected from amine-containing groups, hydroxyl groups, carboxyl groups, carbonyl groups, and combinations thereof. In a further preferred embodiment, the reactive group is an amino group.

Aminated polymeric materials useful in practicing the present invention can be readily produced through a number of methods well known in the art. For example, amines may be introduced into a preformed polymer by plasma treatment of materials with ammonia gas as found in Holmes and Schwartz, Composites Science and Technology, 38: 1-21 (1990). Alternatively, amines can be provided by grafting acrylamide to the polymer followed by chemical modification to introduce amine moieties by methods well known to those skilled in the art; e.g., by the Hofmann rearrangement reaction. Also, grafted acrylamide-containing polymer may be prepared by radiation grafting as set forth in U.S. Pat. No. 3,826,678 to Hoffman et al. A grafted N-(3-aminopropyl)methacrylamide-containing polymer may be prepared by ceric ion grafting as set forth in U.S. Pat. No. 5,344,455 to Keogh et al. Polyvinylamines or polyalkylimines also can be covalently attached to polyurethane surfaces according to the method taught by U.S. Pat. No. 4,521,564 to Solomone et al. Alternatively, for example, aminosilane may be attached to the surface as set forth in U.S. Pat. No. 5,053,048 to Pinchuk.

In yet another embodiment, a polymeric coating material, or a precursor material, is exposed to a high frequency plasma with microwaves or, alternatively, to a high frequency plasma combined with magnetic field support to yield the desired reactive surfaces bearing at least a substantial portion of reactant amino groups upon the substrate to be derivatized with the RNAi agent.

A functionalized carrier or coating surface can be prepared by, for example, first submitting a carrier coating component to a chemical oxidation step. This chemical oxidation step is then followed, for example, by exposing the oxidized substrate to one or more plasma gases containing ammonia and/or organic amine(s) which react with the treated surface. In one embodiment, the gas is selected from the group consisting of ammonia, organic amines, nitrous oxide, nitrogen, and combinations thereof. The nitrogen-containing moieties derived from this gas are preferably selected from amino groups, amido groups, urethane groups, urea groups, and combinations thereof, more preferably primary amino groups, secondary amino groups, and combinations thereof. In another aspect of this embodiment, the nitrogen source is an organic amine. Examples of suitable organic amines include, but are not limited to, methylamine, dimethylamine, ethylamine, diethylamine, ethylmethylamine, n-propylamine, allylamine, isopropylamine, n-butylamine, n-butylmethylamine, n-amylamine, n-hexylamine, 2-ethylhexylamine, ethylenediamine, 1,4-butanediamine, 1,6-hexanediamine, cyclohexylamine, n-methylcyclohexylamine, ethyleneimine, and the like. In a further aspect, the chemical oxidation step is supplemented with, or replaced by, submitting the polymer to one or more exposures to plasma-gas that contains oxygen. In yet a further preferred embodiment, the oxygen-containing plasma gas further contains argon (Ar) gas to generate free radicals. Immediately after a first-step plasma treatment with oxygen-containing gases, or oxygen/argon plasma gas combinations, the oxidized polymer is preferably functionalized with amine groups. As mentioned above, functionalization with amines can be performed with plasma gases such as ammonia, volatile organic amines, or mixtures thereof.

In an exemplary embodiment utilizing ammonia and/or organic amines, or mixtures thereof, as the plasma gases, a frequency in the radio frequency (RF) range of from about 13.0 MHz to about 14.0 MHz is used. A generating power of from 0.1 Watts per square centimeter to about 0.5 Watts per square centimeter of surface area of the electrodes of the plasma apparatus is preferably utilized. An exemplary plasma treatment includes evacuating the plasma reaction chamber to a desired base pressure of from about 10 to about 50 mtorr. After the chamber is stabilized to a desired working pressure, ammonia and/or organic amine gases are introduced into the chamber. Preferred flow rates are typically from about 200 to about 650 standard mL per minute. Typical gas pressure ranges from about 0.01 to about 0.5 Torr, and preferably from about 0.2 to about 0.4 Torr. A current having the desired frequency and level of power is supplied by means of electrodes from a suitable external power source. Power output is up to about 500 Watts, preferably from about 100 to about 400 Watts. The plasma treatment can be performed by means of a continuous or batch process.

Optimization procedures for the plasma treatment and the effect of these procedures on the characteristics and the performance of the reactive polymers can be determined by, for example, evaluating the extent of substrate functionalization. Methods for characterizing functionalized polymers are well known in the art.

The result of the above-described exemplary methods is preferably a polymeric surface that contains a significant number of primary and/or secondary amino groups. These groups are preferably readily reactive at room temperature with an inherent, or an appended, reactive functional group on the RNAi agents. Once the amine-containing polymeric carrier coating is prepared, it can be used to covalently bind the RNAi agents using a variety of functional groups including, for example, ketones, aldehydes, activated carboxyl groups (e.g. activated esters), alkyl halides and the like.

Synthesis of specific RNAi agent/carrier conjugates is generally accomplished by: 1) providing a carrier or coating component comprising an activated polymer, such as an acrylic acid, and an RNAi agent having a position thereon which will allow a linkage to form; 2) reacting the complementary substituents of the RNAi agent and the carrier coating component in an inert solvent, such as methylene chloride, chloroform or DMF, in the presence of a coupling reagent, such as 1,3-diisopropylcarbodiimide or any suitable dialkyl carbodiimide (Sigma Chemical), and a base, such as dimethylaminopyridine, diisopropyl ethylamine, pyridine, triethylamine, etc. Alternative specific syntheses are readily accessible to those of skill in the art (see, for example, Greenwald et al., U.S. Pat. No. 5,880,131).

One skilled in the art understands that in the synthesis of compounds useful in practicing the present invention, one may need to protect various reactive functionalities on the starting compounds and intermediates while a desired reaction is carried out on other portions of the molecule. After the desired reactions are complete, or at any desired time, normally such protecting groups will be removed by, for example, hydrolytic or hydrogenolytic means. Such protection and deprotection steps are conventional in organic chemistry. One skilled in the art is referred to Protective Groups in Organic Chemistry, McOmie, ed., Plenum Press, NY, N.Y. (1973); and, Protective Groups in Organic Synthesis, Greene, ed., John Wiley & Sons, NY (1981) for the teaching of protective groups which may be useful in the preparation of compounds of the present invention.

Delivery of RNAi Agents

The RNAi expression constructs and RNAi agents of the present invention may be introduced into the target cells in vitro or ex vivo and then subsequently placed into a patient to affect therapy, or administered directly to a patient by in vivo administration. Target cells can be obtained from cord blood, bone marrow, peripheral blood or any other method for obtaining stem cells known in the art.

The most common transfection reagents are charged lipophilic compounds that are capable of crossing cell membranes. When these are complexed with an RNAi agent they can act to carry the RNAi agent across the cell membrane. A large number of such compounds are available commercially. Polyethylenimine (PEI) is a class of transfection reagents, chemically distinct from lipophilic compounds that act in a similar fashion to lipophilic compounds, but have the advantage they can also cross nuclear membranes. An example of such a reagent is ExGen 500 (Fermentas). A construct or synthetic gene according to the present invention may be packaged as a linear fragment within a synthetic liposome or micelle for delivery into the target cell.

Another delivery method useful for the method of this invention comprises the use of Cyclosert™ technology as described in U.S. Pat. No. 6,509,323 to Davis et. al. Cyclosert™ technology platform is based upon cup-shaped cyclic repeating molecules of glucose known as cyclodextrins. The “cup” of the cyclodextrin molecule can form “inclusion complexes” with other molecules, making it possible to combine the Cyclosert™ polymers with other moieties to enhance stability or to add targeting ligands. In addition, cyclodextrins have generally been found to be safe in humans (individual cyclodextrins currently enhance solubility in FDA-approved oral and IV drugs) and can be purchased in pharmaceutical grade on a large scale at low cost. These polymers are extremely water soluble, non-toxic and non-immunogenic at therapeutic doses, even when administered repeatedly. The polymers can easily be adapted to carry a wide range of small-molecule therapeutics at drug loadings that can be significantly higher than liposomes.

Compositions may also be injected by microinjection or intramuscular jet injection (for example as described by Furth et al., Anal. Biochem., 205: 265-368, (1992)). Another route of administration is hydrodynamic in which an aqueous formulation of the naked genetic construct, agent or synthetic gene is prepared, usually with a DNase inhibitor, and administered to the vascular system of the patient.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. The active compounds for transmucosal or transdermal administration are are formulated into ointments, salves, gels, or creams as generally known in the art.

The compounds can also be administered by transfection or infection using methods known in the art, including but not limited to the methods described in McCaffrey et al, Nature 418:38-39, 2002 (hydrodynamic transfection); Xia et al, Nature Biotechnol, 20:1006-1010, (2002) (viral-mediated delivery); or Putnam, Am. J. Health Syst. Pharm. 53:151-160, (1996), erratum at Am. J. Health Syst. Pharm. 53:325, (1996)).

The compounds can also be administered by any method suitable for administration of nucleic acid agents. These methods include, but are not limited to gene guns, bio injectors, microencapsulation and skin patches as well as needle-free methods such as the micro-particle DNA vaccine technology disclosed in U.S. Pat. No. 6,194,389 and the mammalian transdermal needle-free vaccination with powder-form vaccine as disclosed in U.S. Pat. No. 6,168,587.

In one embodiment, the nucleic acid agents of this invention are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The techniques for delivery of DNA and RNA constructs to animal cells described in U.S. Pat. Nos. 5,985,847 and 5,922,687 are also applicable. The entire contents of these two specifications are incorporated herein by reference.

Poration technologies, use high-frequency pulses of energy, in a variety of forms (such as radio frequency radiation, laser, heat or sound) to temporarily disrupt the stratum corneum. It is important to note that unlike iontophoresis, the energy used in poration technologies is not used to transport the drug across the skin, but facilitates its movement. Poration provides a “window” through which drug substances can pass much more readily and rapidly than they would normally.

The RNAi agents described herein may be co-administered with one or more other compounds or molecules or administered in conjunction with another treatment modality. By “co-administered” is meant simultaneous administration in the same formulation or in two different formulations via the same or different routes or sequential administration by the same or different routes. By “sequential” administration is meant a time difference of from seconds, minutes, hours or days between the administrations of the two types of molecules. These molecules may be administered in any order. More particularly the present invention contemplates co-administration of a genetic construct in accordance with the present invention with one or more known methods of stem cell manipulation such culture with various cell growth media including cytokines and the like.

While the present invention has been described with reference to specific embodiments, it should be understood by those skilled in the art that various changes may be made and equivalents may be substituted without departing from the true spirit and scope of the invention. In addition, many modifications may be made to adapt a particular situation, material or process to the objective, spirit and scope of the present invention. All such modifications are intended to be within the scope of the invention.

All references cited herein are to aid in the understanding of the invention, and are incorporated in their entireties for all purposes without limitation.

### REFERENCES

Fire, et al., Nature, 391:806-11 (1998);
Tuschl, et al., Genes and Dev., 13:3191-97 (1999);
Elbashir, et al., Nature, 411:494-498 (2001);
U.S. Pat. No. 6,573,099);
Kurreck, Eur. J. Bioch. 270:1628-44 (2003);
Harborth, et al., Antisense Nucleic Acid Drug Rev. 13(2): 83-105 (2003);
Maniatis, Fritsch & Sambrook, Molecular Cloning: A Laboratory Manual (1982);
DNA Cloning: A Practical Approach, Volumes I and II (D. N. Glover, ed. 1985);
Oligonucleotide Synthesis (M. J. Gait, ed. 1984);
Nucleic Acid Hybridization (B. D. Hames & S. J. Higgins, eds. (1984));
Animal Cell Culture (R. I. Freshney, ed. 1986);
RNA Viruses: A practical Approach, (Alan, J. Cann, Ed., Oxford University Press, 2000);
Sambrook et al., Molecular Cloning: A Laboratory Manual, 2nd Ed., Cold Spring Harbor Press, Cold Spring Harbor, N.Y. (1989);
For example, U.S. Pat. No. 6,620,805;
U.S. Pat. No. 6,673,611;
U.S. Publ. Nos. 2004/0171570, 2004/0171032, and 2004/0171031;
U.S. Publ. No. 2004/0161844;
U.S. Publ. No. 2004/0161844;
U.S. Publ. No. 2004/0161777;
U.S. Publ. No. 2004/0147470;
U.S. Publ. No. 2004147023;
U.S. Publ. No. 2004/0147022;
Higashibata, et al., J. Bone Miner. Res. January 19(1):78-88 (2004);
Hoggatt, et al., Circ. Res., December 91(12):1151-59 (2002);
Sohal, et al., Circ. Res. July 89(1):20-25 (2001);
Zhang, et al., Genome Res. January 14(1):79-89 (2004);
see Maraia, et al., Nucleic Acids Res 22(15):3045-52 (1994);
Maraia, et al., Nucleic Acids Res 24(18):3552-59 (1994);
WO 03/022052 A1;
US 2002/0162126 A1;
Xia et al., Nucleic Acids Res 31-17 (2003);
Myers and Miller (1988);
Pearson and Lipman (1988);
Karlin and Altschul (1993);
(Nakai, et al., J. Virol. 76:11343-349 (2002);
(Kay, et al., Nature. 424: 251 (2003) and Thomas, et al., Nature Reviews, Genetics 4:346-58 (2003);
(Tomar et al., Oncogene. 22: 5712-15 (2003);
(Boden, et al., J. Virol. 77(21): 115231-35 (2003);
(Mingozzi, et al., J. Virol. 76(20): 10497-502 (2002);
Thomas, et al., J. Virol. (in press);
Grimm et al. (Blood. 2003-02-0495);
(see Yant, et al., Nature Biotech. 20:999-1004 (2002));
(see Perri, et al, J. Virol. 74(20):9802-07 (2002);
(Antonchuk);
(Kyba PNAS 2003 vol 100);
(Nakajima Embo 2001 20 6836-6844);
U.S. Pat. No. 5,784,164 to Caldwell;
Holmes and Schwartz, Composites Science and Technology, 38:1-21 (1990);
U.S. Pat. No. 5,344,455 to Keogh;
U.S. Pat. No. 3,826,678 to Hoffman;
U.S. Pat. No. 4,521,564 to Solomone et al.;
U.S. Pat. No. 5,053,048 to Pinchuk;
Greenwald et al., U.S. Pat. No. 5,880,131);
Protective Groups in Organic Chemistry, McOmie, ed., Plenum Press, NY, N.Y. (1973); and, Protective Groups in Organic Synthesis, Greene, ed., John Wiley & Sons, NY (1981);
Furth et al., Anal. Biochem., 205: 265-368, (1992);
Xia et al, Nature Biotechnol, 20:1006-1010, (2002);
Putnam, Am. J. Health Syst. Pharm. 53:151-160, 1996, erratum at Am. J Health Syst. Pharm. 53:325, 1996;
U.S. Pat. No. 6,194,389, and the mammalian transdermal needle-free
in U.S. Pat. No. 4,522,811;
U.S. Pat. No. 6,168,587;
U.S. Pat. No. 5,985,847;
U.S. Pat. No. 5,922,687;
Elwood N. J., Cancer Control 2004 voll 11 no. 2:77-85;
Antonchuk J, Sauvageau G, Humphries R K. Exp Hematol. 2001 September; 29(9):1125-34;
T W Austin, et al. Blood Vol 89, 10 p 3624-2625 (1997);
Ingles-Esteve, J. et al. J. Biol. Chem. Vol 276, 48 (2001) p 44873-44880;

What is claimed is:

1. A method for promoting cell growth and inhibiting differentiation in a subject or cell culture, said method comprising administering an RNAi agent to the subject or cell culture, wherein the RNAi agent delays, represses, or otherwise reduces the expression of one or more transcriptionally active genetic regions that are directly or indirectly associated with the differentiation and proliferation of stem cells.

2. The method of claim 1, wherein the one or more transcriptionally active genetic regions encode for one or more proteins selected from the group consisting of WNT2 (Homo sapiens), Wnt2 (Mus musculus), Wnt4 (Mus musculus), WNT4 (Homo sapiens), WIF1 (Homo sapiens), Wnt2 (Rattus norvegicus), Wif1 (Mus musculus), Wif1 (Rattus norvegicus), Wnt3a (Mus musculus), WNT3A (Homo sapiens), Tcf1 (Rattus norvegicus), TCF1 (Homo sapiens), Tcf1 (Mus musculus), Tcf7 (Mus musculus), Tcf3 (Mus musculus), TCF3 (Homo sapiens), TCF7L1 (Homo sapiens), TCF4 (Homo sapiens), Tcf4 (Mus musculus), Tcf4 (Rattus norvegicus), TP53 (Homo sapiens), Hoxb4 (Mus musculus), HOXB4 (Homo sapiens, HOXB3 (Homo sapiens), Notch1 (Drosophila) and (Rattus norvegicus), NOTCH1 (Drosophila) and (Homo sapiens), Notch1 (Drosophila) and (Mus musculus), NOTCH2 (Drosophila) and (Homo sapiens), NOTCH3 (Drosophila) and (Homo sapiens), Notch2 (Drosophila) and (Mus musculus), Notch2 (Drosophila) and (Rattus norvegicus), Notch3 (Drosophila) and (Mus musculus), NOTCH4 (Drosophila) and (Homo sapiens), Terf1 (Mus musculus), TERF2 (Homo sapiens), Tert2 (Mus musculus), TERF1 (Homo sapiens), POT1 (S. pombe) and (Homo sapiens), Pot1 (Mus musculus), CSF3 (Homo sapiens), Csf3 (Mus musculus), STAT3 (Homo sapiens), CSF3R (Homo sapiens), Csf3r (Mus musculus), MYC (Homo sapiens), TERT (Homo sapiens), MYCBP (Homo sapiens), MAD (Homo sapiens), Mad (Mus musculus), TERC (Homo sapiens), TEP1 (Homo sapiens) and (Mus musculus), USF1 (Homo sapiens) and (Mus musculus), and SMRT (Homo sapiens) and (Mus musculus).

3. The method of claim 1, wherein the RNAi agent comprises a ddRNAi expression cassette which encodes an RNA molecule comprising a nucleotide sequence which is at least 70% identical to at least part of a nucleotide sequence comprising a stem cell associated genetic target or a derivative, ortholog, or homolog thereof.

4. The method of claim 3, wherein the ddRNAi expression cassette encodes two or more RNA molecules comprising a nucleotide sequence which is at least 70% identical to at least part of a nucleotide sequence comprising a stem cell associated genetic target or a derivative, ortholog, or homolog thereof.

5. The method of claim 1, wherein the RNAi agent comprises a siRNAi which encodes an RNA molecule comprising a nucleotide sequence which is at least 70% identical to at least part of a nucleotide sequence comprising a stem cell associated genetic target or a derivative, ortholog, or homolog thereof.

6. The method of claim 1, wherein the stem cells are selected from the group consisting of hematopoietic stem cells, myoblasts, osteoblasts, neural stem cells, and embryonic stem cells.

7. The method of claim 1, wherein the RNAi agent is administered to the subject or cell culture by a viral delivery system.

8. The method of claim 1, wherein administering the RNAi agent comprises using a bacterial vector.

9. The method of claim 1, wherein administering the RNAi agent comprises using mini-circles.

10. The method of claim 1, wherein administering the RNAi agent comprises delivering the RNAi agent into cells in vitro or ex vivo and then introducing the cells into an animal subject.

11. A genetically modified cell comprising a ddRNAi expression cassette that expresses a ddRNAi agent that delays, represses, or otherwise reduces the expression of one or more transcriptionally active genetic regions that are directly or indirectly associated with the differentiation and proliferation of stem cells.

12. The genetically modified cell of claim 11, wherein the one or more transcriptionally active genetic regions that are directly or indirectly associated with the differentiation and proliferation of stem cells encode for one or more proteins selected from the group consisting of WNT2 (Homo sapiens), Wnt2 (Mus musculus), Wnt4 (Mus musculus), WNT4 (Homo sapiens), WIF1 (Homo sapiens), Wnt2 (Rattus norvegicus), Wif1 (Mus musculus), Wif1 (Rattus norvegicus), Wnt3a (Mus musculus), WNT3A (Homo sapiens), Tcf1 (Rattus norvegicus), TCF1 (Homo sapiens), Tcf1 (Mus musculus), Tcf7 (Mus musculus), Tcf3 (Mus musculus), TCF3 (Homo sapiens), TCF7L1 (Homo sapiens), TCF4 (Homo sapiens), Tcf4 (Mus musculus), Tcf4 (Rattus norvegicus), TP53 (Homo sapiens), Hoxb4 (Mus musculus), HOXB4 (Homo sapiens, HOXB3 (Homo sapiens), Notch1 (Drosophila) and (Rattus norvegicus), NOTCH1 (Drosophila) and (Homo sapiens), Notch1 (Drosophila) and (Mus musculus), NOTCH2 (Drosophila) and (Homo sapiens), NOTCH3 (Drosophila) and (Homo sapiens), Notch2 (Drosophila) and (Mus musculus), Notch2 (Drosophila) and (Rattus norvegicus), Notch3 (Drosophila) and (Mus musculus), NOTCH4 (Drosophila) and (Homo sapiens), Terf1 (Mus musculus), TERF2 (Homo sapiens), Tert2 (Mus musculus), TERF1 (Homo sapiens), POT1 (S. pombe) and (Homo sapiens), Pot1 (Mus musculus), CSF3 (Homo sapiens), Csf3 (Mus musculus), STAT3 (Homo sapiens), CSF3R (Homo sapiens), Csf3r (Mus musculus), MYC (Homo sapiens), TERT (Homo sapiens), MYCBP (Homo sapiens), MAD (Homo sapiens), Mad (Mus musculus), TERC (Homo sapiens), TEP1 (Homo sapiens) and (Mus musculus), USF1 (Homo sapiens) and (Mus musculus), and SMRT (Homo sapiens) and (Mus musculus).
